(19) United States
(12) Patent Application Publication
(10) Pub. No.: US 2009/0163811 A1
(43) Pub. Date: Jun. 25, 2009

(54) ULTRASONOGRAPH

(75) Inventors: Takenori Fukumoto, Kanagawa (JP); Hisashi Hagiwara, Kanagawa (JP); Takao Suzuki, Kanagawa (JP)

Correspondence Address: PEARNE & GORDON LLP, 1801 EAST 9TH STREET, SUITE 1200, CLEVELAND, OH, 44114-3108, US

(73) Assignee: MATSUSHITA ELECTRIC INDUSTRIAL CO., LTD., Osaka (JP)

(21) Appl. No.: 12/293,520

(22) PCT Filed: Mar. 13, 2007
(86) PCT No.: PCT/JP2007/054894

§ 371 Date: Sep. 18, 2008

(30) Foreign Application Priority Data
Mar. 20, 2006 (JP) 2006-076855

(51) Int. Cl. A61B 8/14

(52) U.S. Cl. 600/443

(57) ABSTRACT

The present invention is to provide an ultrasonograph which can accurately measure an IMT value of a blood vessel by using an ultrasonic wave. The ultrasonograph includes an ultrasonic signal emitting unit (1) for emitting an ultrasonic signal, an ultrasonic echo receiving unit (2) for receiving, as a reflected signal, an ultrasonic echo from inside tissue, and converting the ultrasonic echo into an electronic signal, an amplitude information processing unit (5) for processing amplitude information on the ultrasonic echo, a phase information processing unit (6) for processing phase information of the ultrasonic echo, and a boundary detecting unit (7) for determining a distribution of a boundary between a blood flow region and an intima and a boundary between a media and an adventitia on the basis of at least one processing result output from the amplitude information processing unit and at least one processing result output from the phase information processing unit, and an IMT value calculating unit (9) for computing an IMT value of a blood vessel from position information indicative of the position of the boundary between the blood flow region and the intima and position information indicative of the position of the boundary between the media and the adventitia.

### TECHNICAL FIELD OF THE INVENTION

The present invention relates to an ultrasonograph for diagnosing a condition of a blood vessel by using an ultrasonic wave.

### BACKGROUND OF THE INVENTION

As an example of a method of detecting an Intima-Media Thickness (hereinafter simply referred to as “IMT value”) of a carotid artery, i.e., a thickness between an intima and a media of a vascular wall by using an ultrasonic wave, it is well known that the IMT of the carotid artery is measured from a luminance signal indicative of image on an ultrasonic echo coming from the carotid artery and its surrounding tissues on the assumption that the carotid artery has a normal vascular structure (see patent document 1).

The above method however encounters such a problem that, due to the fact that this method relies on an intensity of the ultrasonic echo, a position of a boundary between a blood flow region and an intima of the vascular wall and a position of a boundary between a media and an adventitia of the vascular wall are inaccurately detected when the ultrasonic echo coming from the intima of the vascular wall is low, or when the luminance signal is deteriorated by noises contained in the ultrasonic echo. As a result, the IMT of the carotid artery is inaccurately measured when the ultrasonic echo coming from the intima of the vascular wall is low, or when the luminance signal is deteriorated by noises contained in the ultrasonic echo. Moreover, when the carotid artery has a local pathology such as an atheroma, the position of the boundary between the blood flow region and the intima and the position of the boundary between the media and the adventitia is inaccurately detected on assumption that the carotid artery has a normal vascular structure.

In order to solve this problem, the position of the boundary between the blood flow region and the intima of the vascular wall and the location of the media are detected on the basis of a hardness of tissues calculated from the change of a phase of the ultrasonic echo. The IMT value of the vascular wall is measured from the position of the boundary between the blood flow region and the intima of the vascular wall and the location of the media (e.g. refer to patent document 2). The above-mentioned method however encounters such a problem that, as with the method based on the luminance information, the IMT value of the vascular wall is measured inaccurately when the hardness of tissues has a high level of noises. As a result of the fact that the above-mentioned method focuses only on the hardness of tissues, the position of the boundary between the blood flow region and the intima of the vascular wall and the location of the media, detected on the basis of a hardness of tissues, deviate from those visually recognized by an operator from the luminance information indicative of the intensity of the ultrasonic echo, and tend to be recognized as unnatural and wrong results.

Patent document 1: Japanese Patent Laid-Open Publication H11-318896
Patent document 2: Internationally published 2004/112568 pamphlet

### DISCLOSURE OF THE INVENTION

Problem to be solved by the Invention

In order to solve the above problem, the invention is to provide an ultrasonograph which can accurately measure an IMT value of a vascular wall of a blood vessel in a region of interest by combining the intensity of the ultrasonic echo with information from the property of tissues of an object, and automatically detecting the position of the boundary between the blood flow region and the intima and the position of the boundary between the media and the adventitia, which are closely equal to those determined visually by an operator.

Means for solving the Problem

In order to solve the above problem, an ultrasonograph according to the present invention comprises: an ultrasonic signal emitting unit for emitting at least one ultrasonic signal in a direction from a skin surface of an object toward a blood vessel of the object; an ultrasonic echo receiving unit for receiving an ultrasonic echo from tissues of the object when the ultrasonic signal is emitted by the ultrasonic signal emitting unit, and converting the ultrasonic echo into an electric signal; an amplitude information processing unit for processing amplitude information indicative of an amplitude of the ultrasonic echo along a direction intersecting a central axis of the blood vessel; a phase information processing unit for processing phase information indicative of a phase of the ultrasonic echo along the direction intersecting the central axis of the blood vessel; and a boundary detecting unit for detecting a position of a boundary between a blood flow region of the blood vessel and an intima of the blood vessel and a position of a boundary between a media of the blood vessel and an adventitia of the blood vessel on the basis of at least one processing result outputted from the amplitude information processing unit and at least one processing result outputted from the phase information processing unit.

The ultrasonograph thus constructed can accurately detect the position of the boundary between the blood flow region and the intima and the position of the boundary between the media and the adventitia, without being significantly affected by the change of a luminance value obtained as the intima of the blood vessel, even when the blood vessel has a local pathology such as atheroma. Hence, the position of the boundary between the blood flow region and the intima and the position of the boundary between the media and the adventitia detected and closely equal to those visually recognized from the luminance information obtained on the basis of the intensity of the ultrasonic echo do not recognized as unnatural and wrong results.

The ultrasonograph according to the present invention may further comprise an IMT value calculating unit for calculating an IMT value indicative of a thickness of a vascular wall defined by the intima and the media on the basis of position information indicative of the position of the boundary between the blood flow region and the intima and position information indicative of the position of the boundary between the media and the adventitia detected by the boundary detecting unit.

The ultrasonograph thus constructed can accurately measure an IMT value of the blood vessel.

The blood vessel has a vascular wall defining a blood flow region through which blood flows, the blood flow region being defined by a front vascular wall close to the ultrasonic signal emitting unit and a back vascular wall far from the ultrasonic signal emitting unit in sectional view. The ultrasonograph according to the present invention may further comprise a region determining unit for determining a region of interest covering at least one of the front vascular wall and the back vascular wall. The amplitude information processing unit may be adapted to process the amplitude information of the ultrasonic echo from the region of interest determined by the region determining unit. The phase processing unit may be adapted to process the phase information of the ultrasonic echo from the region of interest determined by the region determining unit.

The ultrasonograph thus constructed can detect the position of the boundary between the blood flow region and the intima and the position of the boundary between the media and the adventitia.

In the ultrasonograph according to the present invention, the boundary detecting unit may be adapted to detect, on the basis of one heart cycle, the position of the boundary between the blood flow region and the intima and the position of the boundary between the media and the adventitia from the processing result outputted from the amplitude information processing unit and the processing result outputted from the phase information processing unit.

The ultrasonograph thus constructed can more accurately detect the position of the boundary between the blood flow region and the intima and the position of the boundary between the media and the adventitia.

In the ultrasonograph according to the present invention, the ultrasonic signal emitting unit may be adapted to emit at least one ultrasonic pulse signal in a direction toward at least one point on a longitudinal axis of the blood vessel. The amplitude information processing unit may be adapted to perform processing on the basis of an ultrasonic echo coming from the direction toward the point on the longitudinal axis of the blood vessel. The phase information processing unit may be adapted to perform processing on the basis of the ultrasonic echo coming from the direction toward the point on the longitudinal axis of the blood vessel. The boundary detecting unit may be adapted to detect the position of the boundary between the blood flow region and the intima and the position of the boundary between the media and the adventitia on the basis of at least one processing result outputted from the amplitude information processing unit and at least one processing result outputted from the phase information processing unit.

The ultrasonograph thus constructed can accurately detect a boundary between a blood flow region and an intima along the longitudinal axis of the blood vessel, and a position of the boundary between the media and the adventitia.

The ultrasonograph according to the present invention, may further comprise a display unit for displaying, as an image, the amplitude information processed by the amplitude information processing unit, the phase information processed by the phase information processing unit, and the position information representing the position of the boundary between the blood flow region and the intima and the position information indicative of the position of the boundary between the media and the adventitia detected by the boundary detecting unit.

The ultrasonograph thus constructed can allow a user to visually recognize the position of the boundary between the blood flow region and the intima and the position of the boundary between the media and the adventitia.

In the ultrasonograph according to the present invention, one of processing results outputted from the amplitude information processing unit may indicate amplitude of an ultrasonic echo coming from a depth direction of the object.

The ultrasonograph thus constructed can more accurately detect the position of the boundary between the blood flow region and the intima and the position of the boundary between the media and the adventitia.

In the ultrasonograph according to the present invention, one of processing results outputted from the amplitude information processing unit may indicate the rate of change of an ultrasonic echo coming from a depth direction of the object.

The ultrasonograph thus constructed can more accurately detect the position of the boundary between the blood flow region and the intima and the position of the boundary between the media and the adventitia.

In the ultrasonograph according to the present invention, one of processing results outputted from the amplitude information processing unit may indicate a hardness of tissues of the object calculated in a depth direction of the object, from the phase information of the ultrasonic echo.

The ultrasonograph thus constructed can more accurately detect the position of the boundary between the media and the adventitia.

In the ultrasonograph according to the present invention, one of processing results outputted from the amplitude information processing unit may indicate a strain of tissues of the object calculated, in a depth direction of the object, from the phase information of the ultrasonic echo on the basis of one heart cycle.

The ultrasonograph thus constructed can more accurately detect the position of the boundary between the media and the adventitia.

In the ultrasonograph according to the present invention, one of processing results outputted from the amplitude information processing unit may indicate a thickness of tissues of the object calculated, in a depth direction of the object, from the phase information of the ultrasonic echo.

The ultrasonograph thus constructed can more accurately detect the position of the boundary between the media and the adventitia.

In the ultrasonograph according to the present invention, one of processing results outputted from the amplitude information processing unit may indicate a moving velocity of tissues of the object calculated, in a depth direction of the object, from the phase information of the ultrasonic echo on the basis of one heart cycle.

The ultrasonograph thus constructed can more accurately detect the position of the boundary between the media and the adventitia.

In the ultrasonograph according to the present invention, the boundary detecting unit may be adapted to determine a region of detection in a depth direction of the object on the basis of at least one processing result outputted from the amplitude information processing unit, and to detect the position of the boundary between the blood flow region and the intima on the basis of at least one processing result outputted from the phase information processing unit in the region of detection.

The ultrasonograph thus constructed can more accurately detect the position of the boundary between the blood flow region and the intima and the position of the boundary between the media and the adventitia.

In the ultrasonograph according to the present invention, the processing result outputted from the amplitude information processing unit may indicate an intensity of the ultrasonic echo. The processing result outputted from the phase information processing unit may indicate a hardness of tissues of the object calculated, in the depth direction of the object, from the phase information of the ultrasonic echo.

The ultrasonograph thus constructed can more accurately detect the position of the boundary between the media and the adventitia.

In the ultrasonograph according to the present invention, the boundary detecting unit may be adapted to determine a region of detection in a depth direction of the object on the basis of at least one processing result outputted from the amplitude information processing unit, and to detect the position of the boundary between the media and the adventitia on the basis of at least one processing result outputted from the phase information processing unit in the region of detection.

The ultrasonograph thus constructed can more accurately detect the position of the boundary between the media and the adventitia.

In the ultrasonograph according to the present invention, at least one processing result outputted from the amplitude information processing unit may indicate an intensity of an ultrasonic echo coming from a depth direction of the object and the rate of change of the intensity of the ultrasonic echo.

The ultrasonograph thus constructed can more accurately detect the position of the boundary between the media and the adventitia.

In the ultrasonograph according to the present invention, at least one processing result outputted from the amplitude information processing unit may be filtered in a depth direction of the object, and then outputted to the boundary detecting unit.

The ultrasonograph thus constructed can more accurately detect the position of the boundary between the blood flow region and the intima and the position of the boundary between the media and the adventitia without being significantly affected by noises contained in the ultrasonic echo.

In the ultrasonograph according to the present invention, at least one processing result outputted from the amplitude information processing unit may be filtered in a depth direction of the object and in a longitudinal direction of the blood vessel, and then outputted to the boundary detecting unit.

The ultrasonograph thus constructed can more accurately detect the position of the boundary between the blood flow region and the intima and the position of the boundary between the media and the adventitia without being significantly affected by noises contained in the amplitude information obtained from the ultrasonic echo.

In the ultrasonograph according to the present invention, at least one processing result outputted from the phase information processing unit may be filtered in a depth direction of the object, and then outputted to the boundary detecting unit.

The ultrasonograph thus constructed can more accurately detect the position of the boundary between the blood flow region and the intima and the position of the boundary between the media and the adventitia without being significantly affected by noises contained in the phase information obtained from the ultrasonic echo.

In the ultrasonograph according to the present invention, at least one processing result outputted from the phase information processing unit may be filtered in a depth direction of the object and in a longitudinal direction of the blood vessel, and then outputted to the boundary detecting unit.

The ultrasonograph thus constructed can more accurately detect the position of the boundary between the blood flow region and the intima and the position of the boundary between the media and the adventitia without being significantly affected by noises contained in the phase information obtained from the ultrasonic echo.

In the ultrasonograph according to the present invention, the position information indicative of the position of the boundary between the blood flow region and the intima and the position information indicative of the position of the boundary between the media and the adventitia detected by the boundary detecting unit may be filtered in a longitudinal direction of the blood vessel, and then outputted to the IMT value calculating unit.

The ultrasonograph thus constructed can accurately measure an IMT value of a blood vessel, avoid a significant deviation of the detected boundary from a boundary determined visually by an operator based on the luminance information obtained from an intensity of the ultrasonic echo, and eliminate the unnatural and wrong results due to the above deviation of these boundary positions.

In the ultrasonograph according to the present invention, the position information indicative of the position of the boundary between the blood flow region and the intima and the position information indicative of the position of the boundary between the media and the adventitia detected by the boundary detecting unit may be filtered in a longitudinal direction of the blood vessel, and then outputted to the display unit

The ultrasonograph thus constructed can accurately measure an IMT value of a blood vessel, avoid a significant deviation of the detected boundary from a boundary determined visually by an operator based on the luminance information obtained from an intensity of the ultrasonic echo, and eliminate the unnatural and wrong results due to the above deviation of these boundaries.

### Advantageous Effect of the Invention

From the above-mentioned characteristics, it is understood that the ultrasonograph according to the present invention can accurately detect a boundary between a blood flow region and a vascular wall, without being affected by the change of a luminance value obtained as an intima of the vascular wall, even when the blood vessel has a local pathology such as atheroma. Furthermore, the position of the boundary between the blood flow region and the intima and the position of the boundary between the media and the adventitia detected and closely equal to those visually recognized from the luminance information obtained on the basis of the intensity of the ultrasonic echo do not recognized as unnatural and wrong results. Consequently, the ultrasonograph according to the present invention can accurately measure an IMT value of a vascular wall of a blood vessel in a region of interest by combining the intensity of the ultrasonic echo with information from the property of tissues of an object, and automatically detecting the position of the boundary between the blood flow region and the intima and the position of the boundary between the media and the adventitia, which are closely equal to those determined visually by an operator.

### BRIEF DESCRIPTION OF THE DRAWINGS

FIG. 1 is a block diagram showing a general construction of an ultrasonograph according to the first embodiment of the present invention.

FIG. 2 is a schematic diagram for explaining an operation of the ultrasonograph according to the first embodiment of the present invention.

FIG. 3 is a schematic diagram for explaining an operation of the ultrasonograph according to the first embodiment of the present invention.

FIG. 4 is a block diagram showing a general construction of an ultrasonograph according to the second embodiment of the present invention.

FIG. 5 is a schematic diagram for explaining an example of operations of the first and second filter units forming part of the ultrasonograph according to the second embodiment of the present invention.

FIG. 6 is a schematic diagram for explaining another example of operations of the first and second filter units forming part of the ultrasonograph according to the second embodiment of the present invention.

FIG. 7 is a schematic diagram for explaining an example of an operation of the third filter unit of the ultrasonograph according to the second embodiment of the present invention.

### EXPLANATION OF THE REFERENCE NUMERALS

1: transmitting unit

2: receiving unit (ultrasonic echo receiving unit)

3: delay/synthesis unit

4: B-mode processing unit

5: amplitude information processing unit

6: phase information processing unit

7: boundary detecting unit

8: region determining unit

9: IMT value calculating unit

10: image synthesizing unit

11: display unit (display means)

20: skin surface of an object to be inspected

25: first filter unit

26: second filter unit

27: third filter unit

28: filter control unit

30: blood vessel

40: region of interest

51, 52: processing result of amplitude information

61, 62: processing result of phase information

71: position information indicative of the position of the boundary between the blood flow region and the intima

72: position information indicative of the position of the boundary between media and adventitia

73: threshold

74: offset

101: ultrasonic probe (ultrasonic signal emitting unit)

120, 121: region in which detection

301: adventitia

302: internal media

304: blood flow region

305: first vascular wall

306: atheroma

310: boundary between blood flow region and intima

311: second vascular wall

320: boundary between media and adventitia

### PREFERRED EMBODIMENTS OF THE INVENTION

Preferred embodiments of the ultrasonograph according to the present invention will be described hereinafter with reference to the drawings.

### First embodiment

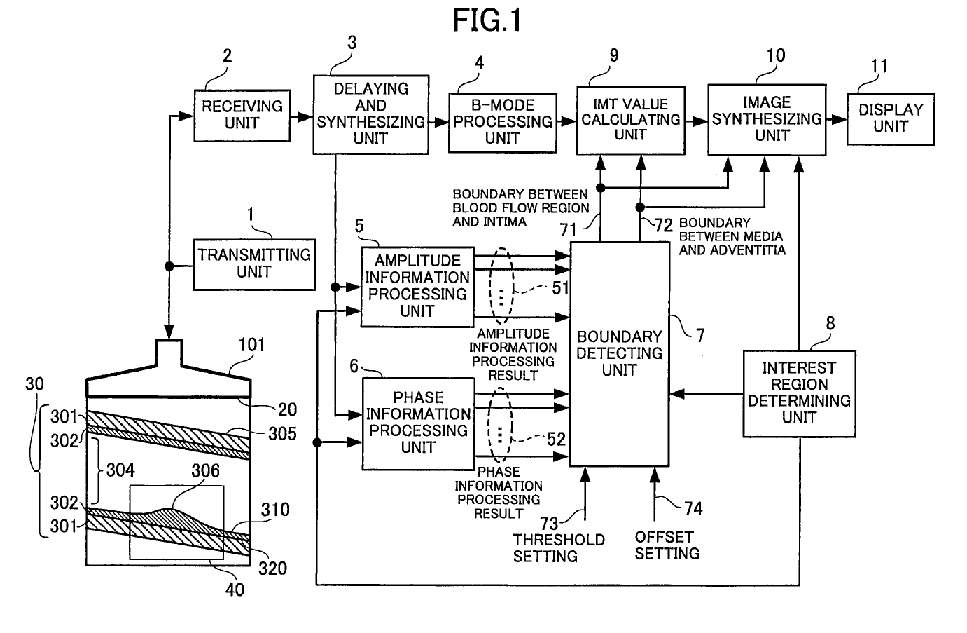

FIG. 1 is a block diagram showing a general construction of an ultrasonograph according to the first embodiment of the present invention. As shown in FIG. 1, the ultrasonograph according to the first embodiment comprises a transmitting unit 1 for generating at least one ultrasonic pulse signal, an ultrasonic probe 101 for emitting the ultrasonic pulse signal generated by the transmitting unit 1 in a direction from a skin surface 20 of an object such as a biological body to a blood vessel 30 inside the object, and receiving an ultrasonic echo from the blood vessel 30 as a reflection of the ultrasonic pulse signal from the blood vessel 30, a receiving unit 2 for converting the ultrasonic echo received by the ultrasonic probe 101 into an ultrasonic echo, a delay/synthesis unit 3 which, if needed, delays and synthesizes the components of the ultrasonic echo produced by the receiving unit 2, a brightness modulation mode processing unit 4 (hereinafter simply referred to as “B-mode processing unit”) for producing sectional image information on the blood vessel 30 from the ultrasonic echo outputted from the delay/synthesis unit 3, an amplitude information processing unit 5 for applying at least one processing to amplitude information indicative of an amplitude of the ultrasonic echo outputted from the delay/synthesis unit 3, and outputting a processing result, and a phase information processing unit 6 for applying at least one processing to phase information indicative of a phase of the ultrasonic echo outputted from the delay/synthesis unit 3, and outputting a processing result. The ultrasonograph according to the first embodiment further comprises a boundary detecting unit 7 for detecting, on the basis of the processing result outputted from the amplitude information processing unit 5 and the processing result outputted from the phase information processing unit 6, a boundary 310 between a blood flow region and an intima and a boundary 320 between a media and an adventitia 301, outputting position information 71 indicative of the position of the boundary 310 between the blood flow region and the intima and position information 72 indicative of the position of the boundary 320 between the media and the adventitia 301, and producing color display image information indicating a sectional image of a blood vessel mapped to a two-dimensional plane, a region determining unit 8 for determining a region of interest (ROI) 40 along a depth direction, i.e., a direction from the skin surface 20 of the object to the blood vessel 30 inside the object to ensure that the amplitude information and the phase information are computed in the region of interest 40, an IMT value calculating unit 9 for calculating an IMT value from the position information 71 indicative of the position of the boundary 310 between the blood flow region and the intima and the position information 72 indicative of the position of the boundary 320 between the media and the adventitia 301, an image synthesizing unit 10 for combining the image information produced by the B-mode processing unit 4 with the image information produced by the boundary detecting unit 7, and a display unit 11 for displaying an image on the basis of the combined image information produced by the image synthesizing unit 10. The ultrasonic probe 101 functions as an ultrasonic signal emitting unit. The receiving unit 2 functions as an ultrasonic echo receiving unit.

In this embodiment, the blood vessel 30 has a vascular wall defining a blood flow region (passageway) 304 through which blood flows. In a longitudinal sectional view, the vascular wall of the blood vessel 30 is defined by a first vascular wall 305 closer to the ultrasonic probe 101, and a second vascular wall 311 far from the ultrasonic probe 101. In this case, the blood vessel 30 defined by the first and second vascular walls 305 and 311 has a local pathological change called “atheroma” 306. The region determining unit 8 is adapted to determine a region of interest 40 along a depth direction, i.e., a direction from the skin surface 20 of the object to the blood vessel 30 inside the object to ensure that the amplitude information and the phase information are computed in the region of interest 40 under the condition that both or either the first vascular wall 305 or the second vascular wall 311 is in the region of interest 40. However, the region of interest 40 may be determined by the region determining unit 8 in cooperation with other devices, or may be determined by an operator's manipulation.

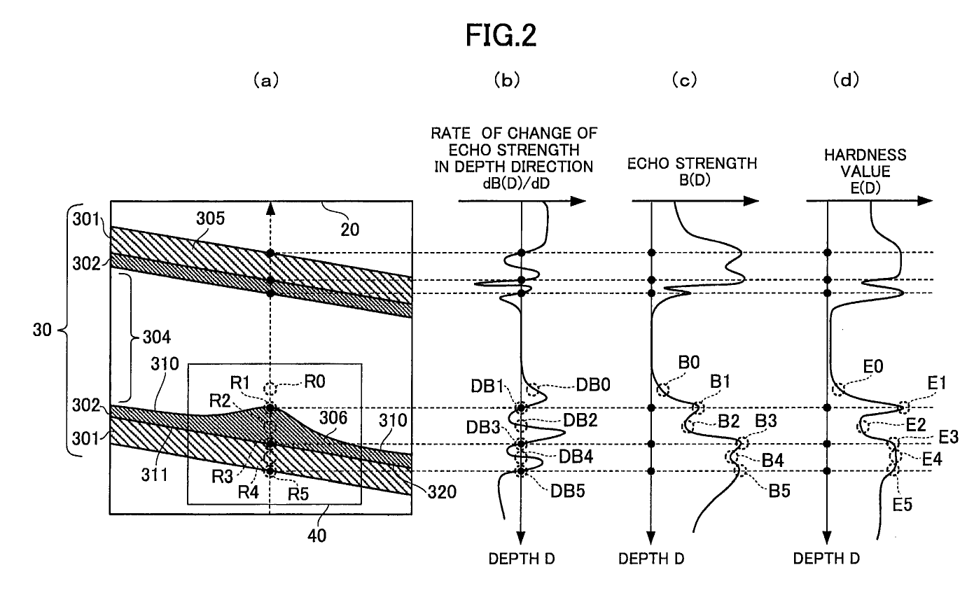

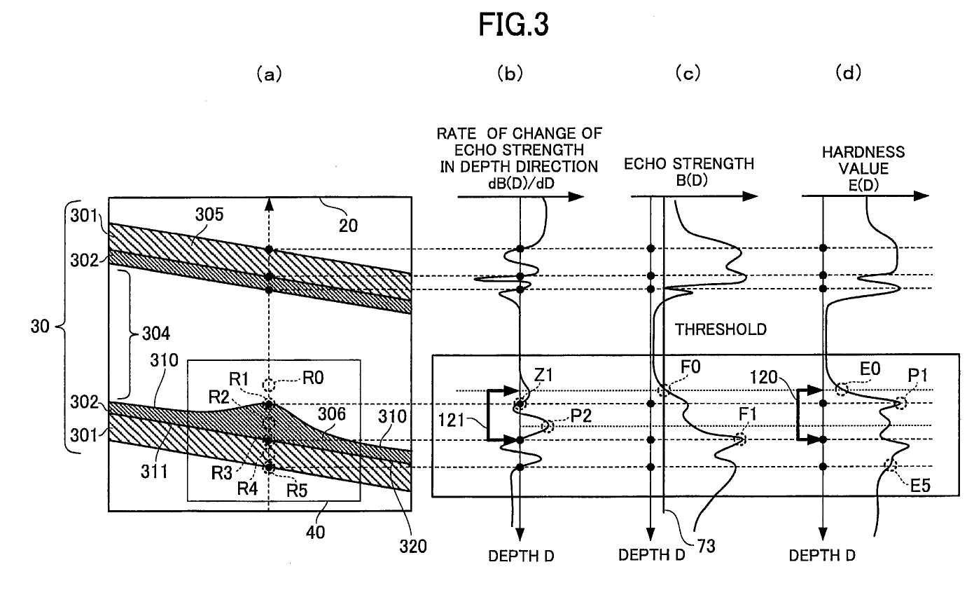

The basic operation of the ultrasonograph according to the first embodiment will be explained hereinafter with reference to FIGS. 2 and 3.

FIG. 2 is a schematic diagram for explaining the position of the boundary 310 between the blood flow region and the intima and the boundary 320 between the media and the adventitia 301. In this case, the boundary 310 between the blood flow region and the intima and the boundary 320 between the media and the adventitia 301 are detected by the boundary detecting unit 7 from an intensity B(D) and the rate of change of the ultrasonic echo in the depth direction dB(D)/dD at a depth D which are computed by the amplitude information processing unit 5 on the basis of the ultrasonic echo received from the blood vessel 30 as a reflection of one emitted ultrasonic pulse signal from the blood vessel 30, and a hardness E(D) of tissues at a depth D which is computed by the phase information processing unit 6 on the basis of the phase of the ultrasonic echo.

The following description is directed to points R0 to R5 defined on a scanning line representing a path of an ultrasonic pulse signal. As shown in FIG. 2(a), the point R0 is within the blood flow region 304 of the blood vessel 30. The point R1 is on a boundary between the second vascular wall 311 and the blood flow region 304. The point R2 is on an atheroma 306 formed in the second vascular wall 311. The point R3 is on a boundary between a media contained in an internal media 302 and an adventitia 301. The point R5 is on a boundary between an adventitia 301 and outside tissues of the blood vessel 30. As shown in FIG. 2(c), it is ideal that the intensity B(D) of the ultrasonic echo has two distinguishable peaks B1 and B2, the peak B1 corresponding to the point R1 indicative of the position of the boundary 310 between the blood flow region and the intima, the peak B3 corresponding to the point R3 indicative of the position of the boundary 320 between the media and the adventitia 301. Then, the location of the peaks can be clearly detected if the blood vessel is normal and if the ultrasonic echo measurement is performed accurately. However, in case of measuring the blood vessel having an atheroma to be diagnosed in detail, or in case of measuring a general blood vessel, the intensity of the ultrasonic echo may lack the above recognizable pair of peaks due to the fact that the intensity of the ultrasonic echo may moderately changed from B0 that corresponds to a blood flow region 304 through B1 which corresponds to the point R1 indicative of the position of the boundary 310 between the blood flow region and the intima, to B2 which corresponds to R2 within the atheroma, or it may contain noises. Therefore, it is impossible to accurately detect the boundary 310 between the blood flow region and the intima and the boundary 320 between the media and the adventitia 301.

On the other hand, as shown in FIG. 2(d), due to the fact that the hardness E(D) of tissues has a peak value E1 which corresponds to the point R1 defined indicative of the position of the boundary 310 between the blood flow region and the intima, and which is significantly more clear than that of the intensity B(D) of the ultrasonic echo, it is possible to accurately detect the boundary 310 between the blood flow region and the intima in comparison with the case of using the intensity B(D) of the ultrasonic echo. However, by taking the method to detect the maximum peak value within the region of interest 40, a separate peak value may result independent of a boundary 310 between the blood flow region and the intima due to the effect of noise occurring as the result of computation of the measured phase of the ultrasonic echo or the hardness E(D) of tissues. This causes a problem of generating a false peak value within the region of interest 40. Also, the hardness E(D) of tissues lacks a clear key property necessary to detect the boundary 320 between the media and the adventitia 301 though the hardness E(D) of tissues has a property that the boundary 310 between the blood flow region and the intima is detectable.

In order to solve the above problems, the ultrasonograph according to the first embodiment restricts, on the basis of the intensity B(D) of the ultrasonic echo, a region in which the boundary 310 between the blood flow region and the intima is detected on the basis of the hardness E(D) of tissues by the boundary detecting unit 7.

The summary of this process will be explained with reference to FIG. 3. The diagram of FIG. 3, similar to that of FIG. 2, shows an intensity B(D) of an ultrasonic echo from tissues, the rate of change of the intensity of the ultrasonic echo dB(D)/dD in depth direction at the depth D computed by the amplitude information processing unit 5, and the hardness E(D) of tissues at the depth D computed by the phase information processing unit 6 from the phase of the ultrasonic echo. Here, a region in which the intensity B(D) of the ultrasonic echo, the rate of change of the intensity of the ultrasonic echo dB(D)/dD, and the hardness E(D) of tissues are computed, corresponding to the region of interest 40, is circumscribed and restricted by a solid line. In this embodiment, the important point is to determine a region corresponding to the region of interest 40 to ensure that the detection of each boundary is performed by the amplitude information processing unit 5 and the phase information processing unit 6 in this region.

Specifically, a region of detection 120, in which the hardness E(D) of tissues in the depth D is obtained by the phase information processing unit 6, is determined through the following process. In FIG. 3 (c), a position at which the intensity B(D) of the ultrasonic echo exceeds a predetermined threshold level 73, and increases with increasing depth from the skin surface in a region circumscribed and restricted by a solid line as a region of interest 40 is determined as a point F0. Next, the positions along the acoustic scanline in which the intensity B(D) of the ultrasonic echo takes the maximum value, the one farthest from the skin surface is searched starting from F0, and set to be F1. Next, within the region between F0 and F1 as shown in FIG. 3 (c), the region corresponding to that in FIG. 3(d) is set as the region of detection 120. Within this region of detection 120, the positions on which the hardness E(D) of tissues takes the maximum values is set to be P1 which corresponds to the point R1 on a boundary 310 between the blood flow region and the intima. As shown in FIG. 1, the above-mentioned threshold 73 can be prescribed from outside.

In this embodiment, when detecting a boundary 310 between the blood flow region and the intima, the hardness E(D) of tissues is used as the processing result outputted from the phase information processing unit 6. The hardness E(D) of tissues is along the body surface to the depth direction computed based on the phase of the ultrasonic echo. However, the invention is not limited to this value. As the processing result outputted from the phase information processing unit 6, any results with a key property that can be used to detect a boundary 310 between the blood flow region and the intima may be used. For example, the followings may be used as the processing result outputted from the phase information processing unit 6. Namely, these are the strain of the tissues of the object along the body surface to the depth direction based on the rate of change in time for one heart cycle, the thickness of the tissues of the object along the body surface to the depth direction, and the moving velocity of the tissues of the object along the body surface to the depth direction based on the rate of change in time for one heart cycle.

In the above explanation, when detecting a boundary 310 between the blood flow region and the intima, the property of the hardness E(D) of tissues at the depth D based on the phase of the ultrasonic echo computed by the phase information processing unit is paid an attention. However, the present invention is not limited to the use of this value. For instance, any value which clearly reveals the characteristic of the tissue of a blood flow different from that of a vascular wall can be used, such as the tissue thickness value, the strain value, or the high frequency component of the velocity. More specifically, any methods which can accurately detect a boundary 310 between the blood flow region and the intima may be used. For example, these methods are: measuring the strain of tissues of the object along a body surface to a depth direction based on the rate of change in time for one heart cycle, measuring the thickness of tissues of the object to be measured along a body surface to the depth direction, and measuring the velocity of the motion of tissues of the object along a body surface to the depth direction based on the rate of change in time for one heart cycle. For instance, in a blood flow region, hardness of tissues is low, but a high frequency component of the velocity of tissues motion is large. On the other hand, in a vascular wall region, hardness of tissues is high, but a high frequency component of the velocity of tissues motion is small. By focusing on such a property, a restriction on the region of detection may be limited according to the high frequency component of the velocity of tissues motion in one heart cycle obtained from the phase information processing unit 6, and within such an region of detection, the locations at which the hardness of tissues takes the maximum value may be detected to be the position information indicative of the position of the boundary between the blood flow region and the intima.

When detecting the boundary 320 between the media and the adventitia 301 as well as the boundary 310 between the blood flow region and the intima, it would be ideal if a clear peak is identified from measured values. Thus, by focusing only on the intensity B(D) of the ultrasonic echo, the simplest method is to take a location at which the intensity B(D) of the ultrasonic echo takes a peak value as the boundary 320 between the media and the adventitia 301, separated from those locations which has already been determined to be the boundary 310 between the blood flow region and the intima. However as stated above, in real measurements, there are many cases in which the boundary 310 between the blood flow region and the intima is unclear. As shown in FIG. 2(c), the intensity B(D) of the ultrasonic echo may have two distinguishable peaks in the ideal boundary 310 between the blood flow region and the intima and the boundary 320 between the media and the adventitia 301, or noises may generate a false peak in B(D). Therefore, it is difficult to accurately detect the boundary 320 between the media and the adventitia 301. Alternatively, it is possible to consider a method to detect the boundary based on the positions at which the rate of change of the intensity B(D) of the ultrasonic echo dB(D)/dD in the depth direction crosses a zero or takes a peak value. However, for the same reasons described above, if only one of B (D) or dB(D)/dD is used, the position of the boundary 320 between the media and the adventitia 301 becomes difficult. Also, the hardness E(D) of tissues does not have clear property that is able to detect the boundary 320 between the media and the adventitia 301.

The ultrasonograph according to the first embodiment detects the position at which the rate of change of the intensity B(D) of the ultrasonic echo dB(D)/dD in a depth direction takes a positive maximum peak value within a region of detection 121 restrictively defined according to the intensity B(D) of the ultrasonic echo, and detects the boundary 320 between the media and the adventitia. This detection enables the boundary 320 between the media and the adventitia 301 to be more accurately detected. This is summarized according to FIG. 3.

More specifically, the region of detection 121 for the rate of change dB(D)/dD along the depth direction of the intensity B(D) of the ultrasonic echo obtained by the amplitude information processing unit 5 is determined by the following steps. In the region of detection 120 indicated in FIG. 2(c) which was determined in order to detect the boundary 310 between the blood flow region and the intima, that is, a region between F0 and F1, the area that corresponds to FIG. 3(b) is prescribed as the region of detection 121. Then, within this region of detection 121, a position at which the rate of change of the intensity B(D) of the ultrasonic echo dB(D)/dD in a depth direction takes the last maximum value is searched in the order from the point F0, and this point of taking the last maximum value is set as the point P2 corresponding to the point R3 indicative of the position of the boundary 320 between the media and the adventitia 301. This process enables the boundary 310 between the blood flow region and the intima and the boundary 320 between the media and the adventitia 301 to be accurately be detected, even if the intensity B(D) of the ultrasonic echo does not have a distinguishable peak in the boundary 310 between the blood flow region and the intima or the boundary 320 between the media and the adventitia 301.

In this explanation, the region of detection is prescribed by focusing on the property of the hardness E(D) of tissues when the boundary detecting unit 7 detects the boundary 320 between the media and the adventitia 301. The invention is not limited to this prescription. Any determining methods that can accurately measure the boundary 320 between the media and the adventitia 301 may be obviously applied to this prescription. For example, the inflection point of the intensity B(D) of the ultrasonic echo may also be used to determine the region of detection, rather than using the intensity B(D) of the ultrasonic echo or its rate of change in a depth direction.

The IMT value calculating unit 9 computes an IMT value from the position information 71 indicative of the position of the boundary 310 between the blood flow region and the intima and the position information 72 indicative of the position of the boundary 320 between the media and the adventitia 301, which are detected by the boundary detecting unit 7. Then, an IMT value calculating unit 9 sends the IMT value to the image synthesizing unit 10. The B-mode processing unit 4 produces the image information representing the cross section of the blood vessel 30 based on the ultrasonic echo which passes through the delay/synthesis unit 3, and submits this image information to the image synthesizing unit 10. The image synthesizing unit 10 then combines the image information provided by the B-mode processing unit 4 with the result provided by the boundary detecting unit 7. Then, the display unit 11 including monitors and other parts displays the image based on the image data combined by the image synthesizing unit 10. When detecting a misalignment between the B-mode image displayed on the display unit 11 and the position of the boundary detected, a predetermined fixed value may be added to or subtracted from the position information 71 indicative of the position of the boundary between the blood flow region and the intima and the position information 72 indicative of the position of the boundary between the media and the adventitia inputted from the boundary detecting unit 7 in order to eliminate the misalignment. More specifically, as shown in FIG. 1, the offset value 74 and the threshold 73 may be set from the outside. In the ultrasonograph according to the embodiment, this setting is performed in the boundary detecting unit 7. However, the invention is not restricted to this setting. Further, the image synthesizing unit 10 displays an IMT value calculated by the IMT value calculating unit 9 if necessary, but the invention is not restricted to such displaying. It may suffice for the display unit 11 to display a boundary that is close to the case in which an operator visually determines such boundary.

Also, due to the fact that the relative position of the ultrasonic probe 101 and the above-mentioned boundaries change in one heart cycle (interval between two R waves), an IMT value varies depending on the timing of calculating an IMT value. However, the IMT value is normally measured during the diastolic when a vascular wall is not contracting. Hence, the timing at which to calculate an IMT value can be determined during the neighborhood of the diastolic in one heart cycle during which an IMT value takes a maximum value. For example, the boundary can be detected very second between one R wave to the next R wave and the distance between the position of the boundary between the blood flow region and the intima and the position of the boundary between the media and the adventitia is calculated occasionally, and the maximum value can be set to be a an IMT value. But, the invention does not restrict the determining method to just this example.

As explained above, the ultrasonograph according to the second embodiment comprises an ultrasonic probe 101 for emitting at least one ultrasonic pulse signal in a direction from a skin surface of an object toward a blood vessel inside the object, and receiving an ultrasonic echo from the blood vessel as a reflection of the ultrasonic pulse signal from the blood vessel, a receiving unit 2 for converting the ultrasonic echo received by the ultrasonic probe 101 into an ultrasonic echo, an amplitude information processing unit 5 for processing amplitude information indicative of an amplitude of the ultrasonic echo coming from a direction intersecting a longitudinal axis of the blood vessel, a phase information processing unit 6 for processing phase information indicative of a phase of the ultrasonic echo coming from a direction intersecting a longitudinal axis of the blood vessel, a boundary detecting unit 7 for detecting, on the basis of at least one processing result outputted from the amplitude information processing unit 5 and at least one processing result outputted from the phase information processing unit 6, a boundary between a blood flow region and an intima and a boundary between a media and an adventitia 301, and an IMT value calculating unit 9 for calculating an IMT value from the position information indicative of the position of the boundary between the blood flow region and the intima and the position information indicative of the position of the boundary between the media and the adventitia 301 detected by the boundary detecting unit 7. Therefore, the ultrasonograph according to the present invention is able to accurately detect the position information indicative of the position of the boundary between the blood flow region and the intima and the position information indicative of the position of the boundary between the media and the adventitia without being significantly affected by the change of a luminance value obtained as an intima of the vascular wall even when the blood vessel has a local pathology such as for example atheroma. Moreover, the configuration in the embodiment enables the detected boundary position not to significantly deviate from the position of each boundary visually recognized from the luminance information obtained on the basis of the intensity of the ultrasonic echo, and do not recognized as unnatural and wrong results. Furthermore, the ultrasonograph according to the present invention can accurately measure an IMT value of a vascular wall of a blood vessel in a region of interest.

### Second Embodiment

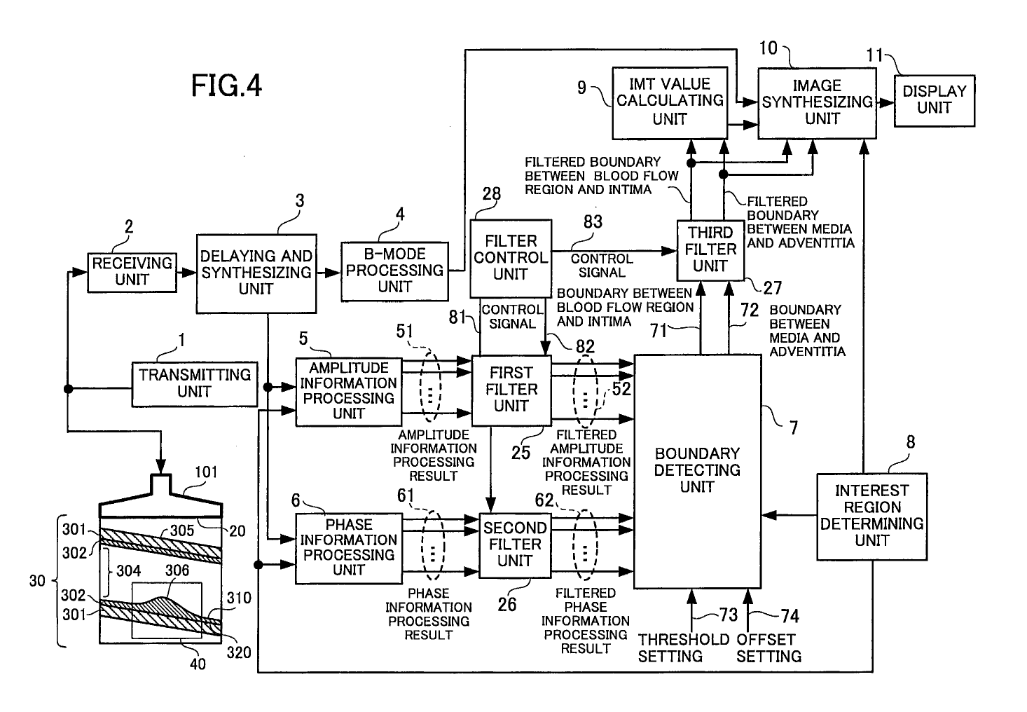

FIG. 4 is a block diagram showing a general construction of an ultrasonograph according to the second embodiment of the present invention. In FIG. 4, elements of the ultrasonograph according to the second embodiment the same as those of the ultrasonograph according to the first embodiment bear the same reference numerals as those of the ultrasonograph according to the first embodiment, and does not explained hereinafter. A method of obtaining position information 71 indicative of the position of the boundary between the blood flow region and the intima and position information 72 indicative of the position of the between the media and the adventitia is the same as a method explained in the first embodiment, and does not explained hereinafter.

As shown in FIG. 4, the ultrasonograph according to the second embodiment comprises, in addition to the constitution of the ultrasonograph according to the first embodiment, a first filter unit 25 for filtering processing results outputted from the amplitude information processing unit 5, and outputting this filtered results to the boundary detecting unit 7, a second filter unit 26 for filtering processing results outputted from the phase information processing unit 6, and outputting this filtered results to the boundary detecting unit 7, a third filter unit 27 for filtering the processing results outputted from the boundary detecting unit 7, and outputting this filtered results to the IMT value calculating unit 9 as well as to the image synthesizing unit 10, and a filter control unit 28 for controlling the first filter unit 25, the second filter unit 26, and the third filter unit 27.

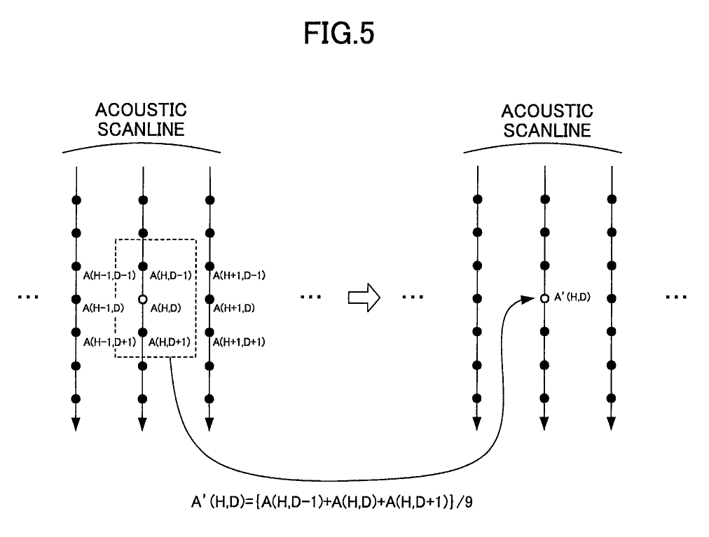

The operation of the ultrasonograph according to the second embodiment will be described hereinafter with reference to FIGS. 5 to 7. The operation of the ultrasonograph according to the second embodiment is substantially the same as that of the ultrasonograph according to the first embodiment with the exception of operations of the first filter unit 25, the second filter unit 26, the third filter unit 27, and the filter control unit 28. Therefore, the operation of the ultrasonograph according to the second embodiment the same as that of the ultrasonograph according to the first embodiment will be not described hereinafter.

The processing results outputted from the amplitude information processing unit 5 and the processing result outputted from the phase information processing unit 6 are filtered in the depth direction from the body surface by the first filter unit 25 and the second filter unit 26, and then outputted to the boundary detecting unit 7.

The following description is directed to an example of a filtering processing to be executed by the first filter unit 25. In FIG. 5, the position of the boundary K(H) is on an acoustic scanline H showing a path of an ultrasonic pulse signal. One of the filtered results outputted to the IMT value calculating unit 9 from the first filter unit 25 is represented by A′(H, D), obtained through step of filtering the amplitude information processing result 51 in a depth direction, and expressed as the following formula.

A′(H, D)={A(H, D−1)+A(H, D)+A(H, D+1)}/3

This example is directed to the simplest weighted mean average filter applied to the first filter unit 25 in which the amplitude information processing result 51 is filtered. Similarly, the phase information processing result 61 is also filtered by the simplest weighted mean average filter applied to the second filter unit 26, and outputted to the boundary detecting unit 7.

In this case, the first filter unit 25 performs the weighted mean average of three amplitude information processing results A(H, D−1), A(H, D), and A(H, D+1). The amplitude information processing result A(H, D) corresponds to a target point, while the amplitude information processing results A(H, D−1) and A(H, D+1) correspond to two neighboring points. The second filter unit 26 performs the weighted mean average of three phase information processing results. However, the present invention is not limited to this case. For example, the number of data needed for the simplest weighted mean average is not limited. The first and second filter units 25 and 26 may be constituted by conventional two dimensional FIR filters, two dimensional non-linear filters, or the like. The first and second filter units 25 and 26 may be operable to remive noises from the amplitude information processing result 51 and the phase information processing result 61. It is obvious that the filter applied to the amplitude information processing result 51 may be different in type or in characteristics from that applied to the phase information processing result 61.

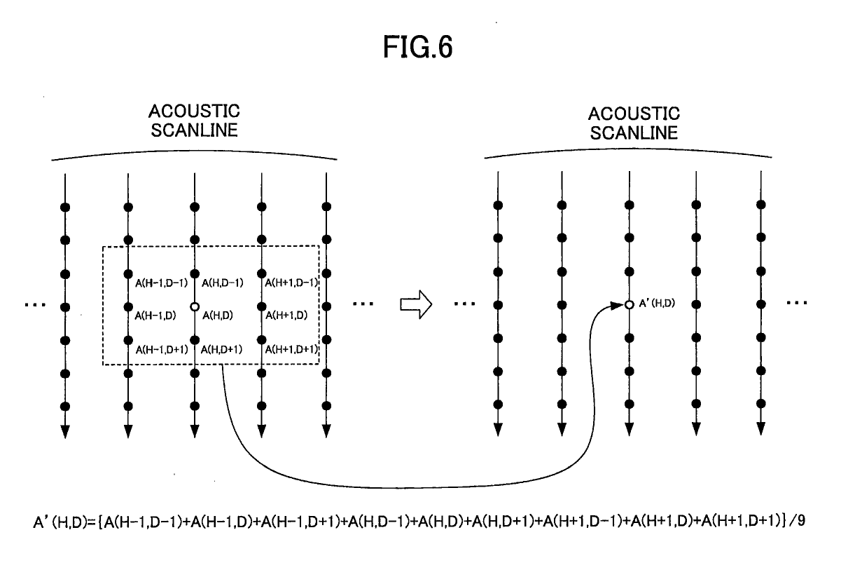

FIG. 6 is a schematic diagram for explaining another example of operations of the first and second filter units forming part of the ultrasonograph according to the second embodiment of the present invention. In FIG. 6, the amplitude information processing result 51 corresponding to a depth D on a particular acoustic scanline H is represented by a function A(H, D), and one filtered result to be outputted from the first filter unit 25 to the boundary detecting unit 7 is represented by a function A″(H, D). When the amplitude information processing result 51 is filtered along a direction of the acoustic scanline H and a depth direction from the skin surface, the filtered result A″(H, D) can be expressed by the following expression.

A″(H, D)={A(H−1, D−1)+A(H−1, D)+A(H−1, D+1)+A(H, D−1)

+A(H, D)+A(H, D+1)+A(H+1, D−1)+A(H+1, D)+A(H+1, D+1)}/9

This example is directed to two dimensional weighted mean average filter applied to the first filter unit 25 as a simple filtering processing. Similarly, the phase information processing result 61 is also filtered by the two dimensional weighted mean average filter applied to the second filter unit 26, and outputted to the boundary detecting unit 7.

In this case, the first filter unit 25 performs the two dimensional weighted mean average of nine amplitude information processing results A(H−1, D−1), A(H−1, D), A(H−1, D+1), A(H, D−1), A(H, D), A(H, D+1), A(H+1, D−1), A(H+1, D), and A(H+1, D+1). The amplitude information processing result A(H, D) corresponds to a target point, while the amplitude information processing results A(H−1, D−1), A(H−1, D), A(H−1, D+1), A(H, D−1), A(H, D+1), A(H+1, D−1), A(H+1, D), and A(H+1, D+1) correspond to eight neighboring points. The second filter unit 26 performs the two dimensional weighted mean average of nine phase information processing results. However, the present invention is not limited to this case. For example, the number of data needed for the two dimensional weighted mean average is not limited. The first and second filter units 25 and 26 may be constituted by conventional two dimensional FIR filters, two dimensional non-linear filters, or the like. The first and second filter units 25 and 26 may be operable to remive noises from the amplitude information processing result 51 and the phase information processing result 61. It is obvious that the filter applied to the amplitude information processing result 51 may be different in type or in characteristics from that applied to the phase information processing result 61.

In the ultrasonograph according thus constructed, the boundary detecting unit 7 can obtain position information 71 indicative of the position of the boundary between the blood flow region and the intima and position information 72 indicative of the position of the boundary between the media and the adventitia without being easily affected from noises contained in the amplitude and phase information processing results obtained from the ultrasonic echo.

The position information 71 indicative of the position of the boundary between the blood flow region and the intima and the position information 72 indicative of the position of the boundary between the media and the adventitia obtained by the boundary detecting unit 7 are outputted to the third filter unit 27, and filtered so that a longitudinal view of a blood vessel detected in a region of interest is displayed on a screen. The function of the third filter unit 27 is to mainly remove noises from the position information 71 indicative of the position of the boundary between the blood flow region and the intima and the position information 72 indicative of the position of the boundary between the media and the adventitia, to remove or reduce the significant deviation of the position of the boundary of the blood vessel detected in a region of interest from a boundary visually recognized by an operator from luminance information based on an intensity of an ultrasonic echo, and to aboid or improve the unnatural and wrong results.

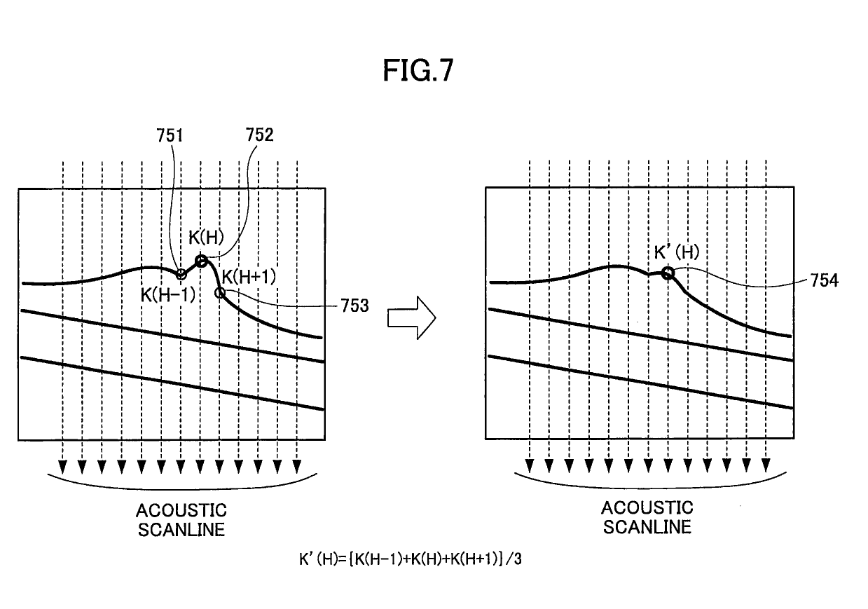

The following description is directed to an example of a filtering processing to be executed by the third filter unit 27. In FIG. 7, the position of the boundary K(H) is on an acoustic scanline H showing a path of an ultrasonic pulse signal. One of the filtered results outputted to the IMT value calculating unit 9 from the third filter unit 28 is represented by K′(H). The filtered result K′(H) is obtained through step of filtering the position information 71 indicative of the position of the boundary between the blood flow region and the intima, and expressed as the following formula.

K′(H)={K(H−1)+K(H)+K(H+1)}/3

This example is directed to the simplest weight mean filter applied to the third filter unit 27 in which the position information 71 indicative of the position of the boundary between the blood flow region and the intima is filtered. Similarly, the position information 72 indicative of the position of the boundary between the media and the adventitia is also filtered by this weight mean filter applied to the third filter unit 27.

As explained above, the ultrasonograph according to the second embodiment comprises an ultrasonic probe 101 for emitting at least one ultrasonic pulse signal in a direction from a skin surface 20 of an object toward a blood vessel 30 inside the object, and receiving an ultrasonic echo from the blood vessel 30 as a reflection of the ultrasonic pulse signal from the blood vessel 30, a receiving unit 2 for converting the ultrasonic echo received by the ultrasonic probe 101 into an ultrasonic echo, an amplitude information processing unit 5 for processing amplitude information indicative of an amplitude of the ultrasonic echo coming from a direction intersecting a longitudinal axis of the blood vessel 30, a phase information processing unit 6 for processing phase information indicative of a phase of the ultrasonic echo coming from a direction intersecting a longitudinal axis of the blood vessel 30, a boundary detecting unit 7 for detecting, on the basis of at least one processing result outputted from the amplitude information processing unit 5 and at least one processing result outputted from the phase information processing unit 6, a boundary 310 between a blood flow region and an intima and a boundary 320 between a media and an adventitia 301, and an IMT value calculating unit 9 for calculating an IMT value from the position information 71 indicative of the position of the boundary 310 between the blood flow region and the intima and the position information 72 indicative of the position of the boundary 320 between the media and the adventitia 301 detected by the boundary detecting unit 7. The ultrasonograph according to the embodiment further comprises a first filter unit 25 for filtering the processing result of the amplitude information processing unit 5, and outputting the filtered result to the boundary detecting unit 7, a second filter unit 26 for filtering the processing result of the amplitude information processing unit 6, and outputting the filtered result to the boundary detecting unit 7, a third filter unit 27 for filtering the processing result of the boundary detecting unit 7, and outputting the filtered result to the IMT value calculating unit 9 and the image synthesizing unit 10, and a filter control unit 28 for controlling the first filter unit 25, the second filter unit 26, and the third filter unit 27. Therefore, the ultrasonograph according to the second embodiment can accurately detect a boundary between a blood flow region and a vascular wall, without being significantly affected by the change of a luminance value obtained as an intima of the vascular wall even when the blood vessel has a local pathology such as for example atheroma. In the ultrasonograph according to the second embodiment, the IMT value calculating unit 9 can accurately calculate an IMT value without being easily affected by noises contained in the position information 71 indicative of the position of the boundary between the blood flow region and the intima and noises contained in the position information 72 indicative of the position of the boundary between the media and the adventitia detected by the boundary detecting unit 7, by reason that noises contained in the position information 71 indicative of the position of the boundary between the blood flow region and the intima and noises contained in the position information 72 indicative of the position of the boundary between the media and the adventitia detected by the boundary detecting unit 7 are removed. The image synthesizing unit 10 can accurately combine the image information produced by the B-mode processing unit 4 with the image information produced by the boudanry dececting unit 7. Therefore, the positions of the detected boundaries are closely equal to those visually recognized from the luminance information obtained on the basis of the intensity of the ultrasonic echo, and do not recognized as unnatural and wrong results. Furthermore, the ultrasonograph according to the present invention can accurately measure an IMT value of a vascular wall of a blood vessel in a region of interest.

Additionally, the third filter unit 27 performs the weighted mean average of three processing results K(H−1), K(H), and K(H+1) as mentioned above. The processing result K(H) corresponds to a target point, while the processing results K(H−1) and K(H+1) correspond to two neighboring points. However, the present invention is not limited to this case. For example, the number of data needed for the weighted mean average is not limited. The third filter unit 27 may be constituted by a conventional FIR filter, a non-linear filter, or the like operable to remove noises contained in the position information 71 indicative of the position of the boundary between the blood flow region and the intima and noises contained in the position information 72 indicative of the position of the boundary between the media and the adventitia detected by the boundary detecting unit 7. It is obvious that the filter applied to the position information 71 indicative of the position of the boundary between the blood flow region and the intima may be different in type or in characteristics from that applied to the position information 72 indicative of the position of the boundary between the media and the adventitia. Further, the filter control unit 28 may set filter coefficients applied to the first filter unit 25, the second filter unit 26 and the third filter unit 27, by cooperating with another device or by an operator's manual controls.

### INDUSTRIAL APPLICABILITY OF THE INVENTION

As will be seen the foregoing description, the ultrasonograph according to the present invention can accurately detect the boundary between the blood flow region 304 and the vascular wall 305, 311 without being significantly affected by the change of a luminance value obtained as an intima even when the blood vessel has a local pathology such as atheroma. Further, the position of the boundary between the blood flow region 304 and the vascular wall 305, 311 and the position of the intima detected in the above-mentioned method do not deviate significantly from these visually determined by an operator on the basis of luminance information obtained from the intensity of the ultrasonic echo, and do not recognized as unnatural and wrong results. Accordingly, the ultrasonograph according to the present invention can accurately measure the IMT value of the vascular wall by combining information obtained from the intensity B(D) of the ultrasonic echo with information extracted from characteristics of tissues, automatically detecting the position of the boundary between the blood flow region 304 and the vascular wall 305, 311 and the location of a media, which are significantly close to those determined visually by an operator, and is useful for diagnosing or displaying an image indicative of a condition of a blood vessel, by using an ultrasonic wave, in the medical field.

1. An ultrasonograph comprising:
an ultrasonic signal emitting unit for emitting at least one ultrasonic signal in a direction from a skin surface of an object toward a blood vessel of said object;
an ultrasonic echo receiving unit for receiving an ultrasonic echo from tissues of said object when said ultrasonic signal is emitted by said ultrasonic signal emitting unit, and converting said ultrasonic echo into an electric signal;
an amplitude information processing unit for processing amplitude information indicative of an amplitude of said ultrasonic echo along a direction intersecting a central axis of said blood vessel;
a phase information processing unit for processing phase information indicative of a phase of said ultrasonic echo along said direction intersecting said central axis of said blood vessel; and
a boundary detecting unit for detecting a position of a boundary between a blood flow region of said blood vessel and an intima of said blood vessel and a position of a boundary between a media of said blood vessel and an adventitia of said blood vessel on the basis of at least one processing result outputted from said amplitude information processing unit and at least one processing result outputted from said phase information processing unit.

2. An ultrasonograph according to claim 1, further comprising:
an IMT value calculating unit for calculating an IMT value indicative of a thickness of a vascular wall defined by said intima and said media on the basis of position information indicative of said position of said boundary between said blood flow region and said intima and position information indicative of said position of said boundary between said media and said adventitia detected by said boundary detecting unit.

3. An ultrasonograph according to claim 1 or claim 2, in which
said blood vessel has a vascular wall defining a blood flow region through which blood flows, said blood flow region being defined by a front vascular wall close to said ultrasonic signal emitting unit and a back vascular wall far from said ultrasonic signal emitting unit in sectional view, and which further comprises:
a region determining unit for determining a region of interest covering at least one of said front vascular wall and said back vascular wall, wherein
said amplitude information processing unit is adapted to process said amplitude information of said ultrasonic echo from said region of interest determined by said region determining unit, and
said phase processing unit is adapted to process said phase information of said ultrasonic echo from said region of interest determined by said region determining unit.

4. An ultrasonograph according to claim 1, wherein
said boundary detecting unit is adapted to detect, on the basis of one heart cycle, said boundary between said blood flow region and said intima and said boundary between said media and said adventitia from said processing result outputted from said amplitude information processing unit and said processing result outputted from said phase information processing unit.

5. An ultrasonograph according to claim 1, wherein
said ultrasonic signal emitting unit is adapted to emit at least one ultrasonic pulse signal in a direction toward at least one point on a longitudinal axis of said blood vessel,
said amplitude information processing unit is adapted to perform processing on the basis of an ultrasonic echo coming from said direction toward said point on said longitudinal axis of said blood vessel,
said phase information processing unit is adapted to perform processing on the basis of said ultrasonic echo coming from said direction toward said point on said longitudinal axis of said blood vessel, and
said boundary detecting unit is adapted to detect said boundary between said blood flow region and said intima and said boundary between said media and said adventitia on the basis of at least one processing result outputted from said amplitude information processing unit and at least one processing result outputted from said phase information processing unit.

6. An ultrasonograph according to claim 1, further comprising:
a display unit for displaying, as an image, said amplitude information processed by said amplitude information processing unit, said phase information processed by said phase information processing unit, and position information indicative of said position of said boundary between said blood flow region and said intima and position information indicative of said position of said boundary between said media and said adventitia detected by said boundary detecting unit.

7. An ultrasonograph according to claim 1, wherein
one of said processing result outputted from said amplitude information processing unit is an amplitude of an ultrasonic echo coming from a depth direction of said object.

8. An ultrasonograph according to claim 1, wherein
one of said processing result outputted from said amplitude information processing unit is the rate of change of an ultrasonic echo coming from a depth direction of said object.

9. An ultrasonograph according to claim 1, wherein
one of said processing result outputted from said amplitude information processing unit is a hardness of tissues of said object calculated in a depth direction of said object, from said phase information of said ultrasonic echo.

10. An ultrasonograph according to claim 1, wherein
one of said processing result outputted from said amplitude information processing unit is a strain of tissues of said object calculated, in a depth direction of said object, from said phase information of said ultrasonic echo on the basis of one heart cycle.

11. An ultrasonograph according to claim 1, wherein
one of said processing result outputted from said amplitude information processing unit is a thickness of tissues of said object calculated, in a depth direction of said object, from said phase information of said ultrasonic echo.

12. An ultrasonograph according to claim 1, wherein
one of said processing result outputted from said amplitude information processing unit is a moving velocity of tissues of said object calculated, in a depth direction of said object, from said phase information of said ultrasonic echo on the basis of one heart cycle.

13. An ultrasonograph according to claim 1, wherein
said boundary detecting unit is adapted to determine a region of detection in a depth direction of said object on the basis of at least one processing result outputted from said amplitude information processing unit, and to detect said boundary between said blood flow region and said intima on the basis of at least one processing result outputted from said phase information processing unit in said region of detection.

14. An ultrasonograph according to claim 13, wherein
said processing result outputted from said amplitude information processing unit is an intensity of said ultrasonic echo, and
said processing result outputted from said phase information processing unit is a hardness of tissues of said object calculated, in said depth direction of said object, from said phase information of said ultrasonic echo.

15. An ultrasonograph according to claim 1, wherein
said boundary detecting unit is adapted to determine a region of detection in a depth direction of said object on the basis of at least one processing result outputted from said amplitude information processing unit, and to detect said boundary between said media and said adventitia on the basis of at least one processing result outputted from said phase information processing unit in said region of detection.

16. An ultrasonograph according to claim 15, wherein
at least one processing result outputted from said amplitude information processing unit is an intensity of an ultrasonic echo coming from a depth direction of said object and the rate of change of said intensity of said ultrasonic echo.

17. An ultrasonograph according to claim 1, wherein
at least one processing result outputted from said amplitude information processing unit is filtered in a depth direction of said object, and then outputted to said boundary detecting unit.

18. An ultrasonograph according to claim 1, wherein
at least one processing result outputted from said amplitude information processing unit is filtered in a depth direction of said object and in a longitudinal direction of said blood vessel, and then outputted to said boundary detecting unit.

19. An ultrasonograph according to claim 1, wherein
at least one processing result outputted from said phase information processing unit is filtered in a depth direction of said object, and then outputted to said boundary detecting unit.

20. An ultrasonograph according to claim 1, wherein
at least one processing result outputted from said phase information processing unit is filtered in a depth direction of said object and in a longitudinal direction of said blood vessel, and then outputted to said boundary detecting unit.

21. An ultrasonograph according to claim 1, wherein
position information indicative of said position of said boundary between said blood flow region and said intima and position information indicative of said position of said boundary between said media and said adventitia detected by said boundary detecting unit are filtered in a longitudinal direction of said blood vessel, and then outputted to said IMT value calculating unit.

22. An ultrasonograph according to claim 6, wherein
said position information indicative of said position of said boundary between said blood flow region and said intima and said position information indicative of said position of said boundary between said media and said adventitia detected by said boundary detecting unit are filtered in a longitudinal direction of said blood vessel, and then outputted to said display unit
